(19) United States
(12) Patent Application Publication
(10) Pub. No.: US 2024/0117340 A1
(43) Pub. Date: Apr. 11, 2024

(54) STRUCTURE TO PREVENT THREADING OF NUCLEIC ACID TEMPLATES THROUGH A NANOPORE DURING SEQUENCING

(71) Applicant: Roche Sequencing Solutions, Inc., Pleasanton, CA (US)

(72) Inventors: Helen Franklin, San Jose, CA (US); Stanislav Fridland, San Francisco, CA (US); Spencer Klugherz, San Jose, CA (US)

(21) Appl. No.: 18/546,216

(22) PCT Filed: Feb. 18, 2021
(86) PCT No.: PCT/EP2021/053946

§ 371 Date: Aug. 11, 2023

(51) Int. Cl. C12N 15/10; C12Q 1/6869; C12Q 1/6876

(52) U.S. Cl. CPC C12N 15/1065; C12Q 1/6869; C12Q 1/6876; C12Q 2600/166

(57) ABSTRACT

The invention related to forming nucleic add templates including control templates for sequencing using a nanopore-based method, wherein the templates of the novel structure disclosed herein are limited or prevented from threading into the nanopore during sequencing.

### FIELD OF THE INVENTION

The invention relates to the field of nucleic add sequencing. More specifically, the invention relates to the field of forming libraries of nucleic aid targets for sequencing.

### BACKGROUND OF THE INVENTION

Nucleic add sequencing using biological and solid-state nanopores is a rapidly growing field, see Ameur, et al. (2019) Single molecule sequencing: towards clinical applications, Trends Biotech., 37:72. In some methods, the nucleic aid template is being threaded through a biological nanopore (U.S. Ser. No. 10/337,060) or a solid-state nanopore (U.S. Ser. No. 10/288,599, US20180038001, U.S. Ser. No. 10/364,507), or a tunneling junction between two electrodes PCT/EP2019/066199 and US20180217083). In other methods, the template is not threaded through nanopore but a detectable moiety (e.g., label or tag) is threaded (U.S. Pat. No. 8,461,854). There are methods to prevent or regulate the rate of threading of the template nucleic add through nanopore. For example, “speed bumps” are complementary oligonucleotides placed outside the pore (U.S. Ser. No. 10/400,278). A tRNA with its trefoil structure can be attached to the template and interact with a “brake” protein to regulate the rate of the template insertion into the pore (U.S. Ser. No. 10/131,944). Hairpins and loops can be present in primers or complementary probes can prevent threading altogether in a non-threading nanopore sequencing method (U.S. Pat. No. 9,605,309). A translating enzyme such as a helicase can also be used to regulate the rate of threading (US20180201993). In solid-state sequencing the template nucleic add is threaded through a pore in a thin solid layer. Such threading can be regulated with a magnetic bead that “stretches” the nucleic add strand (US20190317040).

There is a need for innovative and economic means of controlling or preventing threading of nucleic adds though a biological or solid-state nanopore during sequencing. Ideally, the means would add the smallest number of steps or components to a complex sequencing workflow.

### SUMMARY OF THE INVENTION

The invention relates to the use of a double-hairpin adaptor with a self-priming ability. Especially advantageous for nanopore sequencing, the 5′-hairpin prevents threading of the displaced strand into a nanopore. The invention also includes a method of making a library for nucleic aid sequencing and a control nucleic acid molecule for nanopore sequencing.

A target nucleic add ligated to the novel double-hairpin adaptor so that (at one or both ends), both 5′- and 3′-termini of the resulting nucleic add construct contain hairpin structures. The 3′-hairpin has an extendable end, which acts as a sequencing primer for the first strand. The second strand is displaced during primer extension but both first and second strands retain their hairpins, which prevent threading into a nanopore.

In some embodiments, the invention is an adaptor for nucleic aid libraries comprising a first strand and a second strand wherein: the first strand has a 5′-portion and a 3′-portion, wherein the 5′ portion forms a stem-loop structure having a loop and a stem containing the 5′-end of the first strand, and the 3′-portion comprises a sequence complementary to the second strand; the second strand has a 5′-portion and a 3′-portion, wherein the 3′ portion forms a stem-loop structure having a loop and a stem containing the 3′-end of the second strand, and the 5′-portion comprises a sequence complementary to the first strand, and the first and second strands form a duplex via the 3′-portion of the first strand and the 5′-portion of the second strand. The 3′-portion of the second strand may be extendable by a nucleic aid polymerase. The loop-forming regions may be least 4, 5, 6 and up to 20 or more nucleotides long. The adaptor may comprise one or more molecular barcodes selected from a sample barcode (SID) and a unique molecular identifier barcode (UID). For example, the SID can be located outside the duplex formed by the 3′-portion of the first strand and the 5′-portion of the second strand or the UID can be located within the duplex formed by the 3′-portion of the first strand and the 5′-portion of the second strand. The SID and the UID can comprise a predefined sequence or the UID can be a random sequence.

In some embodiments, the invention is a method of making a library of nucleic adds comprising: attaching to a plurality of double-stranded nucleic acids in a sample a plurality of adaptors, each adaptor comprising the first strand having a 5′-portion and a 3′-portion, wherein the 5′ portion forms a stem-loop structure having a loop and a stem containing the 5′-end of the first strand, and the 3′-portion comprises a sequence complementary to the second strand; the second strand having a 5′-portion and a 3′-portion, wherein the 3′ portion forms a stem-loop structure having a loop and a stem containing an extendable 3′-end of the second strand, and the 5′-portion comprises a sequence complementary to the first strand, and the first and second strands forming a duplex via the 3′-portion of the first strand and the 5′-portion of the second strand. The adaptor may be attached via ligating the duplex formed the 3′-portion of the first strand and the 5′-portion of the second strand to one or both ends of the double-stranded nucleic adds. In some embodiments, prior to attaching, the plurality of nucleic acids is pre-treated to form a blunt end at one or both ends of each nucleic add. The plurality of nucleic adds may be further pre-treated to add one or more non-templated nucleotides to one strand at one or both ends of each nucleic acid. In some embodiments, the duplex formed by the 3′-portion of the first strand and the 5′-portion of the second strand has a single stranded overhang of one or more nucleotides. The adaptor may comprise one or more molecular barcodes such as sample barcode (SID) and a unique molecular identifier barcode (UID). In some embodiments, the number of UIDs in the plurality of adaptors may exceed the number of nucleic adds in the plurality of nucleic adds. In some embodiments, the number of nucleic adds in the plurality of nucleic acids exceeds the number of UIDs in the plurality of adaptors.

In some embodiments, the invention is a method of sequencing nucleic adds in a sample, the method comprising forming a library of nucleic adds as described herein and sequencing the nucleic adds by a sequencing by synthesis method comprising extending the extendable 3′-end of the second strand of the adaptor. The sequencing-by-synthesis method may include detection with a nanopore.

In some embodiments, the invention is a control nucleic acid for use in a sequencing reaction comprising a first strand and a second strand complementary to the first strand and two termini, wherein at least one or the two termini comprises: a 3′-overhang forming a stem-loop structure, wherein the 3′-end of the overhang is extendable by a nucleic aid polymerase, and a 5′-end which upon displacement by the extended 3′-end, forms a stem-loop structure. The loop-forming regions of the loops formed by 3′-ends or the 5′-end is are at least 4, 5, 6 and up to 20 or more nucleotides long.

In some embodiments, the invention is a method of sequencing a library of nucleic adds comprising contacting the library with a control nucleic acid described herein and sequencing the library of nucleic aid by a method including detection with a nanopore.

### BRIEF DESCRIPTION OF THE DRAWINGS

FIG. 1 is a diagram of the sequencing adaptor with two hairpin loops.

FIG. 2 is a diagram of the sequencing adaptor with a single hairpin loop.

### DETAILED DESCRIPTION OF THE INVENTION

### Definitions

Unless defined otherwise, technical and scientific terms used herein have the same meaning as commonly understood by a person of ordinary skill in the art. See, Sambrook et al., Molecular Cloning, A Laboratory Manual, 4^{th} Ed. Cold Spring Harbor Lab Press (2012).

The following definitions are provided to facilitate understanding of the present disclosure.

The term “adaptor” refers to a nucleotide sequence that may be added to another sequence in order to import additional elements and properties to that sequence. The additional elements include without limitation: barcodes, primer binding sites, capture moieties, labels, secondary structures.

The term “barcode” refers to a nucleic add sequence that can be detected and identified. Barcodes can generally be 2 or more and up to about 50 nucleotides long. Barcodes are designed to have at least a minimum number of differences from other barcodes in a population. Barcodes can be unique to each molecule in a sample or unique to the sample and be shared by multiple molecules in the sample. The term “multiplex identifier,” “MID” or “sample barcode” refer to a barcode that identifies a sample or a source of the sample. As such, all or substantially all, MID barcoded polynucleotides from a single source or sample will share an MID of the same sequence; while all, or substantially all (e.g., at least 90% or 99%), MID barcoded polynucleotides from different sources or samples will have a different MID barcode sequence. Polynucleotides from different sources having different MIDs can be mixed and sequenced in parallel while maintaining the sample information encoded in the MID barcode. The term “unique molecular identifier” or “UID,” refer to a barcode that identifies a polynucleotide to which it is attached. Typically, all, or substantially all (e.g., at least 90% or 99%), UID barcodes in a mixture of UID barcoded polynucleotides are unique.

The term “DNA polymerase” refers to an enzyme that performs template-directed synthesis of polynucleotides from deoxyribonucleotides. DNA polymerases include prokaryotic Pol I, Pol II, Pol III, Pol IV and Pol V, eukaryotic DNA polymerase, archaeal DNA polymerase, telomerase and reverse transcriptase. The term “thermostable polymerase,” refers to an enzyme that is stable to heat, is heat resistant, and retains sufficient activity to effect subsequent polynucleotide extension reactions and does not become irreversibly denatured (inactivated) when subjected to the elevated temperatures for the time necessary to effect denaturation of double-stranded nucleic adds. In some embodiments, the following thermostable polymerases can be used: Thermococcus litoralis (Vent, GenBank: AAA72101), Pyrococcus furiosus (Pfu, GenBank: D12983, BAA02362), Pyrococcus woesii, Pyrococcus GB-D (Deep Vent, GenBank: AAA67131), Thermococcus kodakaraensis KODI (KOD, GenBank: BD175553, BAA06142; Thermococcus sp. strain KOD (Pfx, GenBank: AAE68738)), Thermococcus gorgonarius (Tgo, Pdb: 4699806), Sulfolobus solataricus (GenBank: NC002754, P26811), Aeropyrum pernix (GenBank: BAA81109), Archaeglobus fulgidus (GenBank: 029753), Pyrobaculum aerophilum (GenBank: AAL63952), Pyrodictium occultum (GenBank: BAA07579, BAA07580), Thermococcus 9 degree Nm (GenBank: AAA88769, Q56366), Thermococcus fumicolans (GenBank: CAA93738, P74918), Thermococcus hydrothermalis (GenBank: CAC18555), Thermococcus sp. GE8 (GenBank: CAC12850), Thermococcus sp. JDF-3 (GenBank: AX135456; WO0132887), Thermococcus sp. TY (GenBank: CAA73475), Pyrococcus abyssi (GenBank: P77916), Pyrococcus glycovorans (GenBank: CAC12849), Pyrococcus horikoshii (GenBank: NP 143776), Pyrococcus sp. GE23 (GenBank: CAA90887), Pyrococcus sp. ST700 (GenBank: CAC 12847), Thermococcus pacifcus (GenBank: AX411312.1), Thermococcus zilligii (GenBank: DQ3366890), Thermococcus aggregans, Thermococcus barossii, Thermococcus celer (GenBank: DD259850.1), Thermococcus profundus (GenBank: E14137), Thermococcus siculi (GenBank: DD259857.1), Thermococcus thioreducens, Thermococcus onnurineus NAL, Sulfolobus acidocaldarium, Sulfolobus tokodaii, Pyrobaculum calidifontis, Pyrobaculum islandicum (GenBank: AAF27815), Methanococcus jannaschii (GenBank: Q58295), Desulforococcus species TOK, Desulfurococcus, Pyrolobus, Pyrodictium, Staphylothermus, Vulcanisaetta, Methanococcus (GenBank: P52025) and other archaeal B polymerases, such as GenBank AAC62712, P956901, BAAA07579)), thermophilic bacteria Thermus species (e.g., flavus, ruber, thermophilus, lacteus, rubens, aquaticus), Bacillus stearothermophilus, Thermotoga maritima, Methanothermus fervidus, KOD polymerase, TNA1 polymerase, Thermococcus sp. 9 degrees N-7, T4, T7, phi29, Pyrococcus furiosus, P. abyssi, T. gorgonarius, T. litoralis, T. zilligii, T. sp. GT, P. sp. GB-D, KOD, Pfu, T. gorgonarius, T. zilligii, T. litoralis and Thermococcus sp. 9N-7 polymerases. In some cases, the nucleic add (e.g., DNA or RNA) polymerase may be a modified naturally occurring Type A polymerase. A further embodiment of the invention generally relates to a method wherein a modified Type A polymerase, e.g., in a primer extension, end-modification (e.g., terminal transferase, degradation, or polishing), or amplification reaction, may be selected from any species of the genus Meiothermus, Thermotoga, or Thermomicrobium. Another embodiment of the invention generally pertains to a method wherein the polymerase, e.g., in a primer extension, end-modification (e.g., terminal transferase, degradation or polishing), or amplification reaction, may be isolated from any of Thermus aquaticus (Taq), Thermus thermophilus, Thermus caldophilus, or Thermus filiformis. A further embodiment of the invention generally encompasses a method wherein the modified Type A polymerase, e.g., in a primer extension, end-modification (e.g., terminal transferase, degradation, or polishing), or amplification reaction, may be isolated from Bacillus stearothermophilus, Sphaerobacter thermophilus, Dictoglomus thermophilum, or Escherichia coli. In another embodiment, the invention generally relates to a method wherein the modified Type A polymerase, e.g., in a primer extension, end-modification (e.g., terminal transferase, degradation, or polishing), or amplification reaction, may be a mutant Taq-E507K polymerase. Another embodiment of the invention generally pertains to a method wherein a thermostable polymerase may be used to effect amplification of the target nucleic add.

The term “hairpin” refers to a secondary structure formed by a single strand of nucleic add comprising at least one double stranded region (“stem”) and the region forming the stem is interrupted by a single stranded region “loop.” There is no defined size or relative size for the stem and loop regions.

The term “nucleic acid” or “polynucleotide” refers to deoxyribonucleic adds (DNA) or ribonucleic adds (RNA) and polymers thereof in either single- or double-stranded form. Unless specifically limited, the term encompasses nucleic adds containing known analogues of natural nucleotides that have similar binding properties as the reference nucleic add and are metabolized in a manner similar to naturally occurring nucleotides. Unless otherwise indicated, a particular nucleic add sequence also implicitly encompasses conservatively modified variants thereof (e.g., degenerate codon substitutions), alleles, orthologues, SNPs, and complementary sequences as well as the sequence explicitly indicated.

The term “primer” refers to an oligonucleotide, which binds to a specific region of a single-stranded template nucleic add molecule and initiates nucleic add synthesis via a polymerase-mediated enzymatic reaction. Typically, a primer comprises fewer than about 100 nucleotides and preferably comprises fewer than about 30 nucleotides. A target-specific primer specifically hybridizes to a target polynucleotide under hybridization conditions. Such hybridization conditions can include, but are not limited to, hybridization in isothermal amplification buffer (20 mM Tris-HCl, 10 mM (NH₄)₂SO₄), 50 mM KCl, 2 mM MgSO₄, 0.1% TWEEN® 20, pH 8.8 at 25° C.) at a temperature of about 40° C. to about 70° C. In addition to the target-binding region, a primer may have additional regions, typically at the 5′-poriton. The additional region may include universal primer binding site or a barcode.

The term “sample” refers to any biological sample that comprises nucleic add molecules, typically comprising DNA or RNA. Samples may be tissues, cells or extracts thereof, or may be purified samples of nucleic add molecules. The term “sample” refers to any composition containing or presumed to contain target nucleic add. Use of the term “sample” does not necessarily imply the presence of target sequence among nucleic acid molecules present in the sample. The sample can be a specimen of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic adds isolated therefrom. A sample may also include cell-free material, such as cell-free blood fraction that contains cell-free DNA (cfDNA) or circulating tumor DNA (ctDNA). The sample can be collected from a non-human subject or from the environment.

The term “target” or “target nucleic acid” refer to the nucleic aid of interest in the sample. The sample may contain multiple targets as well as multiple copies of each target.

The term “universal primer” refers to a primer that can hybridize to a universal primer binding site. Universal primer binding sites can be natural or artificial sequences typically added to a target sequence in a non-target-specific manner.

Sequencing nucleic acids using biological or solid state nanopores is a rapidly developing field with many technological solutions becoming available (see Ameur, et al. (2019) Single molecule sequencing: towards clinical applications, Trends Biotech., 37:72). One common issue with nanopore sequencing is control of translocation (threading) of the nucleic aid through pore. Some workflows include merely controlling the rate with which the nucleic acid moves through the opening. Solutions include the use of a helicase (US20180201993) or a magnetic particle (US20190317040) to slow down or regulate the rate of translocation. Some structures can be connected to the pore for example, a “speed bump” complementary oligonucleotide (U.S. Ser. No. 10/400,278). Other structures are attached to the template molecule, for example, a tRNA structure attached to the template interacting with a “brake” protein (U.S. Ser. No. 10/131,944) in the pore complex or hairpins present on sequencing primers (U.S. Pat. No. 9,605,309).

Some nanopore sequencing technologies do not involve translocation of a strand through a nanopore. In such technologies, preventing the translocation (threading) altogether is desired. The present invention is suitable for both control and prevention of translocation through the nanopore.

In addition to controlling translocation of the strand being sequenced, a separate problem is the disposal of the complementary strand in the case of a double stranded template. When the double-stranded nucleic acid template is unwound during the sequencing-by-synthesis (SBS) reaction, the non-sequenced strand must be prevented from threading through the nanopore in both threading and non-threading embodiments of nanopore sequencing.

The invention comprises methods and compositions related to forming nucleic acid templates for sequencing and libraries of nucleic acids templates for sequencing. The templates and libraries are suitable for any method of sequencing but have a special advantage for nanopore sequencing.

In one embodiment, the invention is a novel adaptor comprising a double stranded portion for ligation to the nucleic acid to be sequenced. The adaptor further comprises a novel feature of at least one stem-loop or hairpin structure on the end opposite the double stranded portion. In some embodiments, the adaptor has a single stem-loop structure. In other embodiments, the adaptor has two stem-loop structures. The adaptor retains an extendable 3′-end, which can serve as a primer, for example a sequencing primer or an amplification primer. The adaptor may comprise any of the features useful in a sequencing adaptor including barcodes, primer binding sites or capture moieties, e.g., for separation or purification of the adapted nucleic adds. For example, the double stranded portion of the adaptor can comprise a unique molecular barcode (UID) to uniquely mark the adapted nucleic add or a multiplexing sample barcode (SID) or (MID) to identically mark all the nucleic adds in the sample as coming from the same source. The adaptor may comprise a capture moiety such as biotin for capturing the adapted nucleic adds and separating them from non-adapted nucleic adds. The special advantage of the adaptor is the stem-loop or hairpin structure formed by at least one end of each strand in the adapted nucleic add. The size of the loop is sufficient to inhibit or prevent translocation of the strand through a nanopore during sequencing. Depending on the nanopore used, the length of the loop-forming sequence can be 4, 5, 6 and up to 20 or more nucleotides in order to form a loop of sufficient size to prevent or inhibit translocation. One of skill in the art is able to determine the length of the loop forming sequence either experimentally or empirically knowing the sequence and secondary, tertiary of quaternary structure of the nanopore forming protein.

In one embodiment, the invention is a method of making a library of adapted nucleic adds for sequencing. The novel adaptor is attached to one or both ends of each sample nucleic add. The library may be purified or separated from unused adaptors and un-adapted sample nucleic acids.

In yet another embodiment, the invention is a control molecule for sequencing a library of sample nucleic adds. The control molecule has ends with novel structures described herein. In particular, the control nucleic aid molecule comprises a first strand and a second strand complementary to the first strand. The control molecule further comprises two termini, wherein at least one or the two termini comprises: a 3′-overhang forming a stem-loop structure, wherein the 3′-end of the overhang is extendable by a nucleic acid polymerase, and a recessed 5′-end which upon displacement by the extended 3′-end, also forms a stem-loop structure. The size of the loop in the stem-loop structure on both strands is sufficient to inhibit or prevent translocation of the strand through a nanopore during sequencing.

The present invention comprises simultaneous isolation and sequencing of target nucleic adds in a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids (e.g., urine, sputum, serum, plasma or lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, or fecal samples). The sample may comprise whole blood or blood fractions where normal or tumor cells may be present. In some embodiments, the sample, especially a liquid sample may comprise cell-free material such as cell-free DNA or RNA including cell-free tumor DNA or tumor RNA. In some embodiments, the sample is a cell-free sample, e.g., cell-free blood-derived sample where cell-free tumor DNA or tumor RNA are present. In other embodiments, the sample is a cultured sample, e.g., a culture or culture supernatant containing or suspected to contain nucleic acids derived from the cells in the culture or from an infectious agent present in the culture. In some embodiments, the infectious agent is a bacterium, a protozoan, a virus or a mycoplasma.

Target nucleic acids are the nucleic acid of interest that may be present in the sample. Each target is characterized by its nucleic acid sequence. The present invention enables detection of one or more RNA or DNA targets. In some embodiments, the DNA target nucleic add is a gene or a gene fragment (including exons and introns) or an intergenic region, and the RNA target nucleic add is a transcript or a portion of the transcript to which target-specific primers hybridize. In some embodiments, the target nucleic add contains a locus of a genetic variant, e.g., a polymorphism, including a single nucleotide polymorphism or variant (SNP of SNV), or a genetic rearrangement resulting e.g., in a gene fusion. In some embodiments, the target nucleic add comprises a biomarker, i.e., a gene whose variants are associated with a disease or condition. For example, the target nucleic acids can be selected from panels of disease-relevant markers described in U.S. patent application Ser. No. 14/774,518 filed on Sep. 10, 2015. Such panels are available as AVENIO ctDNA Analysis kits (Roche Sequencing Solutions, Pleasanton, Cal.) In other embodiments, the target nucleic acid is characteristic of a particular organism and aids in identification of the organism or a characteristic of the pathogenic organism such as drug sensitivity or drug resistance. In yet other embodiments, the target nucleic acid is a unique characteristic of a human subject, e.g., a combination of HLA or KIR sequences defining the subject's unique HLA or KIR genotype. In yet other embodiments, the target nucleic acid is a somatic sequence such as a rearranged immune sequence representing an immunoglobulin (including IgG, IgM and IgA immunoglobulin) or a T-cell receptor sequence (TCR). In yet another application, the target is a fetal sequence present in maternal blood, including a fetal sequence characteristic of a fetal disease or condition or a maternal condition related to pregnancy.

In some embodiments, the target nucleic add is RNA (including mRNA, microRNA, viral RNA). In other embodiments, the target nucleic add is DNA including cellular DNA or cell-free DNA (cfDNA) including circulating tumor DNA (ctDNA). The target nucleic acid may be present in a short or long form. Longer target nucleic acids may be fragmented. In some embodiments, the target nucleic add is naturally fragmented, e.g., includes circulating cell-free DNA (cfDNA) or chemically degraded DNA such as the one found in chemically preserved or ancient samples.

In some embodiments, the invention comprises a step of nucleic add isolation. Generally, any method of nucleic aid extraction that yields isolated nucleic acids comprising DNA or RNA may be used. Genomic DNA or RNA may be extracted from tissues, cells, liquid biopsy samples (including blood or plasma samples) using solution-based or solid-phase based nucleic add extraction techniques. Nucleic add extraction can include detergent-based cell lysis, denaturation of nucleoproteins, and optionally removal of contaminants. Extraction of nucleic acids from preserved samples may further include a step of deparaffinization. Solution based nucleic aid extraction methods may comprise salting out methods or organic solvent or chaotrope methods. Solid-phase nucleic extraction methods can include but are not limited to silica resin methods, anion exchange methods or magnetic glass particles and paramagnetic beads (KAPA Pure Beads, Roche Sequencing Solutions, Pleasanton, Cal.) or AMPure beads (Beckman Coulter, Brea, Cal.)

A typical extraction method involves lysis of tissue material and cells present in the sample. Nucleic acids released from the lysed cells can be bound to a solid support (beads or particles) present in solution or in a column, or membrane where the nucleic acids may undergo one or more washing steps to remove contaminants including proteins, lipids and fragments thereof from the sample. Finally, the bound nucleic acids can be released from the solid support, column or membrane and stored in an appropriate buffer until ready for further processing. Because both DNA and RNA must be isolated, no nucleases may be used and care should be taken to inhibit any nuclease activity during the purification process.

In some embodiments, the input DNA or input RNA require fragmentation. In such embodiments, RNA may be fragmented by a combination of heat and metal ions, e.g., magnesium. In some embodiments, the sample is heated to 85°−94° C. for 1-6 minutes in the presence of magnesium. (KAPA RNA HyperPrep Kit, KAPA Biosystems, Wilmington, Mass). DNA can be fragmented by physical means, e.g., sonication, using available instruments (Covaris, Woburn. Mass.) or enzymatic means (KAPA Fragmentase Kit, KAPA Biosystems).

In some embodiments, the isolated nucleic add is treated with DNA repair enzymes. In some embodiments, the DNA repair enzymes comprise a DNA polymerase which has 5′-3′ polymerase activity and 3′-5′ single stranded exonuclease activity, a polynucleotide kinase which adds a 5′ phosphate to the dsDNA molecule, and a DNA polymerase which adds a single dA base at the 3′ end of the dsDNA molecule. The end repair/A-tailing kits are available e.g., Kapa Library Preparation, kits including KAPA Hyper Prep and KAPA HyperPlus (Kapa Biosystems, Wilmington, Mass.).

In some embodiments, the DNA repair enzymes target damaged bases in the isolated nucleic adds. In some embodiments, sample nucleic aid is partially damaged DNA from preserved samples, e.g., formalin-fixed paraffin embedded (FFPET) samples. Deamination and oxidation of bases can result in an erroneous base read during the sequencing process. In some embodiments, the damaged DNA is treated with uracil N-DNA glycosylase (UNG/UDG) and/or 8-oxoguanine DNA glycosylase.

In some embodiments, the invention comprises an amplification step. The isolated nucleic adds can be amplified prior to further processing. This step can involve linear or exponential amplification. Amplification may be isothermal or involve thermocycling. In some embodiments, the amplification is exponential and involves PCR. In some embodiments, gene-specific primers are used for amplification. In other embodiments, universal primer binding sites are added to target nucleic aid e.g., by ligating an adaptor comprising the universal primer binding sites. All adaptor-ligated nucleic acids have the same universal primer binding sites and can be amplified with the same set of primers. The number of amplification cycles where universal primers are used can be low but also can be 10, 20 or as high as about 30 or more cycles, depending on the amount of product needed for the subsequent steps. Because PCR with universal primers has reduced sequence bias, the number of amplification cycles need not be limited to avoid amplification bias.

In some embodiments, the invention utilizes an adaptor nucleic acid composed of two strands with the structure described herein. In some embodiments, the adaptor molecules are in vitro synthesized artificial sequences. In other embodiments, the adaptor molecules are in vitro synthesized naturally occurring sequences. In yet other embodiments, the adaptor molecules are isolated naturally occurring molecules or isolated non-naturally occurring molecules.

The double-stranded end of the hairpin adaptor may be ligated to the double stranded nucleic acid molecules. The adaptor may be ligated at one or both ends of the double-stranded molecule.

The adaptor oligonucleotide can have overhangs or blunt ends on the terminus to be ligated to the target nucleic acid. In some embodiments, the novel adaptor described herein comprises blunt ends to which a blunt-end ligation of the target nucleic acid can be applied. The target nucleic acids may be blunt-ended or may be rendered blunt-ended by enzymatic treatment (e.g., “end repair.”). In other embodiments, the blunt-ended DNA undergoes A-tailing where a single A nucleotide is added to the 3′-end of one or both blunt ends. The adaptors described herein are made to have a single T nucleotide extending from the blunt end to facilitate ligation between the nucleic acid and the adaptor. Commercially available kits for performing adaptor ligation include AVENIO ctDNA Library Prep Kit or KAPA HyperPrep and HyperPlus kits (Roche Sequencing Solutions, Pleasanton, CA). In some embodiments, the adaptor ligated DNA may be separated from excess adaptors and unligated DNA.

In some embodiments, the adaptor contains additional features, e.g., a barcode, an amplification primer binding site or a sequencing primer binding site.

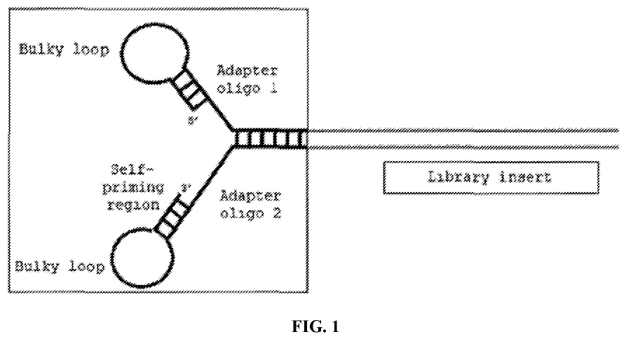

FIG. 1 illustrates the novel adaptor ligated to a nucleic aid of interest. Referring to FIG. 1, the adaptor comprises a first strand (top strand, “Adapter oligo 1”) and a second strand (bottom strand, “Adapter oligo 2”). The adaptor comprises a double-stranded region of hybridization between the first and second adaptor strands (“Adaptor oligos”). This region is capable of being ligated to a target nucleic add (“library insert”). Each strand also comprises a hairpin region having a stem and a loop. In reference to the adaptor illustrated in FIG. 1, the adaptor is ligated to a target nucleic acid on one end and one remaining unligated 5′-end and one remaining unligated 3′-end. The first (top) strand contains the 5′-end, which is non-extendable and is in the double-stranded stem part of the top stem-loop structure. The second (bottom) strand contains the 3′-end. The 3′-end is in the double-stranded stem part of the bottom stem-loop structure. The 3′-end is extendable and can serve as a primer to copy the bottom strand of the target nucleic aid without the need for a separate primer. In this embodiment, the 3′-end of the adaptor is a self-priming region. The self-priming region acts as a sequencing primer or an amplification primer.

In some embodiments, the adaptor-ligated nucleic aid is sequenced after adaptor ligation. In other embodiments, the adaptor-ligated target nucleic add is amplified prior to sequencing. Each one of the copy strands will contain adaptor sequences capable of folding in the double-hairpin structure shown in FIG. 1. The size of the loop is sufficient to inhibit or prevent translocation of either strand through a nanopore during sequencing. The length of the loop-forming region may be designed to be at least 3, 4, 5, 6 and up to 20 or more nucleotides long, depending on the nanopore being used in sequencing.

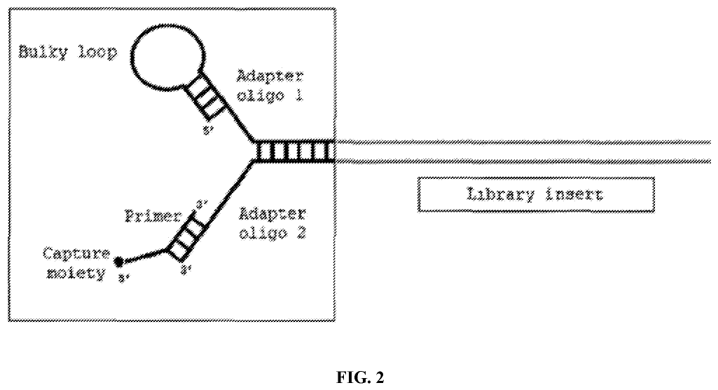

FIG. 2 illustrates a different embodiment of the novel adaptor ligated to a nucleic add of interest. Referring to FIG. 2, the adaptor comprises a first strand (top strand, “Adapter oligo 1”) and a second strand (bottom strand, “Adapter oligo 2”). The adaptor comprises a double-stranded region of hybridization between the first and second adaptor strands (“Adaptor oligos”). This region is capable of being ligated to a target nucleic add (“library insert”). The first (top) strand comprises a hairpin region having a stem and a loop. The second (bottom) strand comprises a capture moiety. In the embodiment shown in FIG. 2, the capture moiety is present in a third oligonucleotide hybridized to the 3′-end of the bottom adaptor strand.

A capture moiety may be any moiety capable of specifically interacting with another capture molecule. Capture moieties—capture molecule pairs include avidin (streptavidin)—biotin, antigen—antibody, magnetic (paramagnetic) particle—magnet, or oligonucleotide—complementary oligonucleotide. The capture molecule can be bound to a solid support so that any nucleic aid on which the capture moiety is present is captured on solid support and separated from the rest of the sample or reaction mixture. In some embodiments, the capture molecule comprises a capture moiety for a secondary capture molecule. For example, a capture moiety may be an oligonucleotide complementary to a capture oligonucleotide (capture molecule). The capture oligonucleotide may be biotinylated and captured on a streptavidin bead.

In some embodiments, the adaptor-ligated nucleic add is enriched via capturing the capture moiety and separating the adaptor-ligated target nucleic adds from unligated nucleic adds in the sample.

In some embodiments, the third oligonucleotide hybridized to the 3′-end of the bottom adaptor strand (FIG. 2) serves as a sequencing primer or an amplification primer. In some embodiments, the extension product of the third oligonucleotide is captured via the capture moiety. Capture of the extension product separates the extension product from unligated sample nucleic adds and optionally, from the target nucleic adds strands not having the capture moiety as well.

In some embodiments, the stem portion of the adaptor includes a modified nucleotide increasing the melting temperature of the capture oligonucleotide, e.g., 5-methyl cytosine, 2,6-diaminopurine, 5-hydroxybutynl-2′-deoxyuridine, 8-aza-7-deazaguanosine, a ribonucleotide, a 2′O-methyl ribonucleotide or a locked nucleic add. In another aspect, the capture oligonucleotide is modified to inhibit digestion by a nuclease, e.g., by a phosphorothioate nucleotide.

In some embodiments, the invention involves an amplification step, e.g., prior to ligating novel adaptors described herein. The primers may be target-specific. A target specific primer comprises at least a portion that is complementary to the target. If additional sequences are present, such as a barcode or a second primer binding site, they are typically located in the 5′-portion of the primer. The target may be a gene sequence (coding or non-coding) or a regulatory sequence present in RNA such as an enhancer or a promoter. The target may also be an intergenic sequence. In other embodiments, the primers are universal, e.g., can amplify all nucleic adds in the sample regardless of the target sequence. Universal primers anneal to universal primer binding sites added to the nucleic adds in the sample by extending a primer having the universal primer binding site or by ligating an adaptor, including the adaptor having the novel structures described herein.

In some embodiments, the invention utilizes a barcode. Detecting individual molecules typically requires molecular barcodes such as described in U.S. Pat. Nos. 7,393,665, 8,168,385, 8,481,292, 8,685,678, and 8,722,368. A unique molecular barcode is a short artificial sequence added to each molecule in the patient's sample typically during the earliest steps of in vitro manipulations. The barcode marks the molecule and its progeny. The unique molecular barcode (UID) has multiple uses. Barcodes allow tracking each individual nucleic add molecule in the sample to assess, e.g., the presence and amount of circulating tumor DNA (ctDNA) molecules in a patient's blood in order to detect and monitor cancer without a biopsy (Newman, A., et al., (2014) An ultrasensitive method for quantitating circulating tumor DNA with broad patient coverage, Nature Medicine doi:10.1038/nm.3519).

A barcode can be a multiplex sample ID (MID) used to identify the source of the sample where samples are mixed (multiplexed). The barcode may also serve as a unique molecular ID (UID) used to identify each original molecule and its progeny. The barcode may also be a combination of a UID and an MID. In some embodiments, a single barcode is used as both UID and MID. In some embodiments, each barcode comprises a predefined sequence. In other embodiments, the barcode comprises a random sequence. In some embodiments of the invention, the barcodes are between about 4-20 bases long so that between 96 and 384 different adaptors, each with a different pair of identical barcodes are added to a human genomic sample. A person of ordinary skill would recognize that the number of barcodes depends on the complexity of the sample (i.e., expected number of unique target molecules) and would be able to create a suitable number of barcodes for each experiment.

Unique molecular barcodes can also be used for molecular counting and sequencing error correction. The entire progeny of a single target molecule is marked with the same barcode and forms a barcoded family. A variation in the sequence not shared by all members of the barcoded family is discarded as an artifact and not a true mutation. Barcodes can also be used for positional deduplication and target quantification, as the entire family represents a single molecule in the original sample (Newman, A., et al., (2016) Integrated digital error suppression for improved detection of circulating tumor DNA, Nature Biotechnology 34:547).

In some embodiments, the number of UIDs in the plurality of adaptors may exceed the number of nucleic adds in the plurality of nucleic acids. In some embodiments, the number of nucleic adds in the plurality of nucleic adds exceeds the number of UIDs in the plurality of adaptors.

In some embodiments, the invention is a library of target nucleic adds formed as described herein. The library comprises double-stranded nucleic add molecules comprising nucleic acid targets present in the original sample. The nucleic acid molecules of the library further comprise novel adaptors described herein at one or both ends of the target nucleic add sequence. The library nucleic adds may comprise additional elements such as barcodes and primer binding sites. In some embodiments, the additional elements are present in adaptors and are added to the library nucleic adds via adaptor ligation. In other embodiments, some or all of the additional elements are present in amplification primers and are added to the library nucleic adds prior to adaptor ligation by extension of the primers. The amplification may be linear (including only one round of extension) or exponential, e.g., Polymerase Chain Reaction (PCR). In some embodiments, some additional elements are added by primer extension while the remaining additional elements are added by adaptor ligation.

The utility of adaptors and amplification primers for introducing additional elements in to a library of nucleic adds to be sequenced has been described e.g., in U.S. Pat. Nos. 9,476,095, 9,260,753, 8,822,150, 8,563,478, 7,741,463, 8,182,989 and 8,053,192.

In some embodiments, the invention further comprises a step of enriching for desired target nucleic adds. The desired nucleic adds can be enriched prior to forming a library according to the novel library forming method of described herein. Alternatively, the enrichment can take place after eh library is formed, i.e., on the molecules of the library.

In some embodiments, the method utilizes a pool of target-specific oligonucleotide probes (e.g., capture probes). The enrichment can be by subtraction in which case, capture probes are complementary to an abundant undesired sequences including ribosomal RNA (rRNA) or abundantly expressed genes (e.g., globin). In the case of subtraction, the undesired sequences are captured by the capture probes and removed from the mixture of target nucleic acids or the library of nucleic adds and discarded. For example, the capture probes may comprise a binding moiety that can be captured on solid support.

In other embodiments, the enrichment is capture and retention in which case, capture probes are complementary to one or more target sequences. In this case the target sequences are captured by the capture probes from the mixture of target nucleic adds or the library of nucleic adds and retained while the remainder of the solution is discarded.

For enrichment, the capture probes may be free in solution or fixed to solid support. The prp0bes can be produced and amplified e.g., by the method described in the U.S. Pat. No. 9,790,543. The probes may also comprise a binding moiety (e.g., biotin) and be capable of being captured on solid support (e.g., avidin or streptavidin containing support material).

In some embodiments, the invention comprises intermediate purification steps. For example, any unused oligonucleotides such as excess primers and excess adaptors are removed, e.g., by a size selection method selected from gel electrophoresis, affinity chromatography and size exclusion chromatography. In some embodiments, size selection can be performed using Solid Phase Reversible Immobilization (SPRI) technology from Beckman Coulter (Brea, Cal.). In some embodiments, a capture moiety (FIG. 2) is used to capture and separate adaptor-ligated nucleic adds from unligated nucleic adds or primer extension products from the template strands.

The nucleic adds and libraries of nucleic adds formed as described herein or amplicons thereof can be subjected to nucleic add sequencing. Sequencing can be performed by any method known in the art. Especially advantageous is the high-throughput single molecule sequencing method utilizing nanopores. In some embodiments, the nucleic adds and libraries of nucleic adds formed as described herein are sequenced by a method involving threading through a biological nanopore (U.S. Ser. No. 10/337,060) or a solid-state nanopore (U.S. Ser. No. 10/288,599, US20180038001, U.S. Ser. No. 10/364,507). In other embodiments, sequencing involves threading tags through a nanopore. (U.S. Pat. No. 8,461,854) or any other presently existing or future DNA sequencing technology utilizing nanopores.

In some embodiments, the sequencing step involves sequence analysis. In some embodiments, the analysis includes a step of sequence aligning. In some embodiments, aligning is used to determine a consensus sequence from a plurality of sequences, e.g., a plurality having the same barcodes (UID). In some embodiments barcodes (UIDs) are used to determine a consensus from a plurality of sequences all having an identical barcode (UID). In other embodiments, barcodes (UIDs) are used to eliminate artifacts, i.e., variations existing in some but not all sequences having an identical barcode (UID). Such artifacts resulting from PCR errors or sequencing errors can be eliminated.

In some embodiments, the number of each sequence in the sample can be quantified by quantifying relative numbers of sequences with each barcode (UID) in the sample. Each UID represents a single molecule in the original sample and counting different UIDs associated with each sequence variant can determine the fraction of each sequence in the original sample. A person skilled in the art will be able to determine the number of sequence reads necessary to determine a consensus sequence. In some embodiments, the relevant number is reads per UID (“sequence depth”) necessary for an accurate quantitative result. In some embodiments, the desired depth is 5-50 reads per UID.

### EXAMPLES

### Example 1. Novel Adaptors for Nucleic Acid Sequencing

The sequencing adaptor is composed of two oligonucleotides (adaptor oligo 1 and adaptor oligo 2) which possess a complementary portion allowing them to form a partially double-stranded adaptor. Adapter oligo 1 further contain DNA sequined at the 5′-end which result in intramolecular stem-loop structure to form under sequencing reaction conditions. The size of the loop structure can be adjusted with DNA length and sequence composition to minimize potential of the displaced 5′-end threading through the sequencing nanopore. Adaptor oligo 2 also contains a stem-loop forming structure at the 3′-end and a free 3′-end for the nucleic add polymerase to bind. This 3;-end is extended during amplification or sequencing with the adaptor oligo 1-ligated strand being eventually displaced by the sequencing or amplification polymerase, the 5′-end loop limiting threading of the displace strand through the sequencing nanopore.

### Example 2. Forming a Control Nucleic Acid for Nanopore Sequencing

A DNA insert containing the desired endonuclease sites, the DNA primer annealing sites and self-complementary end sequences is first cloned in a plasmid vector. The plasmid is grown in a host bacterium; the plasmid is extracted and purified. The plasmid is then digested with an endonuclease that provides a blunt cut, for example PmeI, in order to linearize the plasmid. The linearized plasmid is further digested with a nicking endonuclease, for example, Nt.BbvCI to provide a short excised single stranded fragment at the 5′ends of the linearized plasmid. The plasmid is further denatured and cooled in the presence of an oligo that is complementary to the excised fragment. The excised fragment hybridizes to the complementary oligo, which may be biotin labeled to allow removal of the hybridized excised fragment using streptavidin bead purification. Excision of the 5′-ends of the plasmid allows the 3′-end to form a secondary structure such as a hairpin (stem-loop) that prevents threading of the single stranded end through a nanopore during sequencing of the DNA. The nascent 5′-ends of the plasmid are similarly designed so that on displacement during polymerase extension of a primer oligo annealed to the single stranded regions of the 3′-ends, the 5′-ends also form a secondary structure to prevent threading of the displaced 5′-ends through the nanopore.

1. An adaptor for nucleic acid libraries, wherein the adaptor comprises a first strand and a second strand, and wherein:
(a) the first strand has a 5′-portion and a 3′-portion, wherein the 5′-portion of the first strand forms a stem-loop structure, wherein the stem-loop structure comprises a loop and a stem containing the 5′-end of the first strand, and wherein the 3′-portion of the first strand comprises a sequence complementary to the second strand;
(b) the second strand has a 5′-portion and a 3′-portion, wherein the 3′-portion of the second strand forms a stem-loop structure having, wherein the stem-loop structure comprises a loop and a stem containing the 3′-end of the second strand, and wherein the 5′-portion of the second strand comprises a sequence complementary to the first strand; and
(c) the first strand and the second strand form a duplex via the 3′-portion of the first strand and the 5′-portion of the second strand.

2. The adaptor of claim 1, wherein the 3′-portion of the second strand is extendable by a nucleic acid polymerase.

3. The adaptor of claim 1, wherein one or both of the loop regions is at least 4, 5, 6, and up to 20 or more nucleotides long.

4. The adaptor of claim 1, wherein the adaptor comprises one or more molecular barcodes.

5. The adaptor of claim 4, wherein the one or more molecular barcodes is selected from a sample barcode (SID) and a unique molecular identifier barcode (UID).

6. The adaptor of claim 5, wherein the SID is located outside the duplex formed by the 3′-portion of the first strand and the 5′-portion of the second strand.

7. The adaptor of claim 5, wherein the UID is located within the duplex formed by the 3′-portion of the first strand and the 5′-portion of the second strand.

8. The adaptor of claim 5, wherein the SID and the UID comprise a predefined sequence gr a random sequence.

9. A method of making a library of nucleic acids, wherein the method comprises: attaching to a plurality of double-stranded nucleic acids in a sample a plurality of adaptors, and wherein each adaptor comprises:
(a) a first strand having a 5′-portion and a 3′-portion, wherein the 5-portion of the first strand forms a stem-loop structure, wherein the stem-loop structure comprises a loop and a stem containing the 5′-end of the first strand, and wherein the 3′-portion of the first strand comprises a sequence complementary to a second strand;
(b) the second strand having a 5′-portion and a 3′-portion, wherein the 3′-portion of the second strand forms a stem-loop structure, wherein the stem-loop structure comprises a loop and a stem containing an extendable 3′-end of the second strand, and wherein the 5′-portion of the second strand comprises a sequence complementary to the first strand; and
(c) the first strand and second strand forming a duplex via the 3′-portion of the first strand and the 5′-portion of the second strand.

10. The method of claim 9, wherein the adaptor is attached via ligating the duplex formed by the 3′-portion of the first strand and the 5′-portion of the second strand to one or both ends of the double-stranded nucleic acids.

11. The method of claim 10, wherein prior to attaching, the plurality of nucleic acids is pre-treated to form a blunt end at one or both ends of each nucleic acid.

12. The method of claim 9, wherein the duplex formed by the 3′-portion of the first strand and the 5′-portion of the second strand has a single-stranded overhang of one or more nucleotides.

13. A method of sequencing nucleic acids in a sample, wherein the method comprises: forming a library of nucleic acids according to claim 9, and sequencing the library of nucleic acids by a sequencing by synthesis method comprising extending the extendable 3′-end of the second strand of the adaptor.

14. A control nucleic acid for use in a sequencing reaction, wherein the control nucleic acid comprises: (i) a first strand and a second strand, wherein the second strand is complementary to the first strand; and (ii) two termini, wherein at least one gf the two termini comprises:
(a) a 3′-overhang forming a stem-loop structure, wherein the 3′-end of the 3′-overhang is extendable by a nucleic acid polymerase; and
(b) a 5′-end, which, upon displacement by the extended 3′-end, forms a stem-loop structure.

15. A method of sequencing a library of nucleic acids comprising: (i) contacting the library of nucleic acids with a control nucleic acid according to claim 14, and (ii) sequencing the library of nucleic acids by a method, wherein the method includes detection with a nanopore.
